(19) United States
(12) Plant Patent Application Publication
(10) Pub. No.: US 2024/0397842 P1
(43) Pub. Date: Nov. 28, 2024

(54) Trollius Plant Named 'MACTRO010'

(50) ;

(71) Applicants: Alasdair MacGregor, Kirkcudbright (GB); Elizabeth MacGregor, Kirkcudbright (GB); John Alasdair, Kirkcudbright (GB)

(72) Inventors: Alasdair MacGregor, Kirkcudbright (GB); Elizabeth MacGregor, Kirkcudbright (GB); John Alasdair, Kirkcudbright (GB)

(21) Appl. No.: 18/831,056

(22) Filed: May 22, 2024

(30) Foreign Application Priority Data
May 22, 2023 (QZ) 2023/1086

(51) Int. Cl. A01H 6/72

(52) U.S. Cl. CPC A01H 6/72
USPC PLT/263.1

(57) ABSTRACT

‘MACTRO010’ is a new and distinctive variety of Trollius plant which is characterized by large, cyanthiform flowers held high above the dark green trifoliate foliage, a relatively large number of tepals with a light cream yellow general coloration, light yellow tepaloids, and light yellow pistils and stamens. The new variety propagates successfully by stem cuttings and tissue culture and has shown to be uniform and stable in the resulting generations from asexual propagation.

### CROSS REFERENCE TO RELATED APPLICATIONS

This application claims priority to the Community Plant Variety Rights application number 2023/1086, filed May 22, 2023, which is herein incorporated by reference.

Latin name of the genus and species: The Latin name of the genus and species of the novel variety disclosed herein is Trollius L.

Variety denomination: The inventive variety of Trollius disclosed herein has been given the variety denomination ‘MACTRO010’.

### BACKGROUND OF THE INVENTION

Parentage: ‘MACTRO010’ is an openly-pollination seedling selection of Trollius x cultorum ‘Cheddar’ (not patented) discovered by the inventors at their commercial nursery in Kirkcudbright, Scotland, in the spring of 2010. The male parent is unknown. Seeds were harvested from a cultivated population of ‘Cheddar’ plants, then germinated, and the resulting seedlings were then grown to a mature size in nursery containers. In 2010, the inventors discovered one seedling that exhibited large, pale cream yellow flowers. The plant was isolated and planted into the ground for further evaluation in order to confirm the distinctness and uniformity of the characteristics first observed. After approximately five years of evaluation, this new and distinctive cultivar was selected as a new cultivar.

Asexual Reproduction: Asexual reproduction of ‘MACTRO010’ was first accomplished in 2011 by way of vegetative division in Kirkcudbright, Scotland, and subsequently by way of meristematic tissue culture micro propagation at a laboratory in Heerhugowaard, the Netherlands in approximately 2016. Several successive generations have shown that the unique features of the instant cultivar are stable and reproduce true to type.

### SUMMARY OF THE INVENTION

The cultivar ‘MACTRO010’ has not been observed under all possible environmental conditions and the phenotype may vary somewhat with variations in the instant environment such as temperature, day length, and light intensity, without, however, any variance in genotype. The following traits have been repeatedly observed and represent the distinguishing characteristics of the new Trollius cultivar, ‘MACTRO010’.
1. Trollius ‘MACTRO010’ exhibits dark green trifoliate basal foliage that gives rise to flowering stems which bear large, cyanthiform flowers held high above the foliage; and
2. Trollius ‘MACTRO010’ exhibits flowers with perianths comprised of multiple whorls of relatively large, upright and inwardly-cupped tepals which envelope a single whorl of upright, linear tepaloids which themselves ring the pistils and stamens; and
3. Trollius ‘MACTRO010’ exhibits a relative abundance of tepals with a light cream yellow general coloration; and
4. Trollius ‘MACTRO010’ exhibits tepaloids, pistils, and stamens with a yellow general coloration.

### BRIEF DESCRIPTION OF THE FIGURES

FIG. 1 shows, as nearly true as it is reasonably possible to make the same in color illustrations of this type, an exemplary plant of ‘MACTRO010’ grown in a commercial greenhouse in the Netherlands. This plant is approximately 15-months-old, shown planted in a 19 cm container.

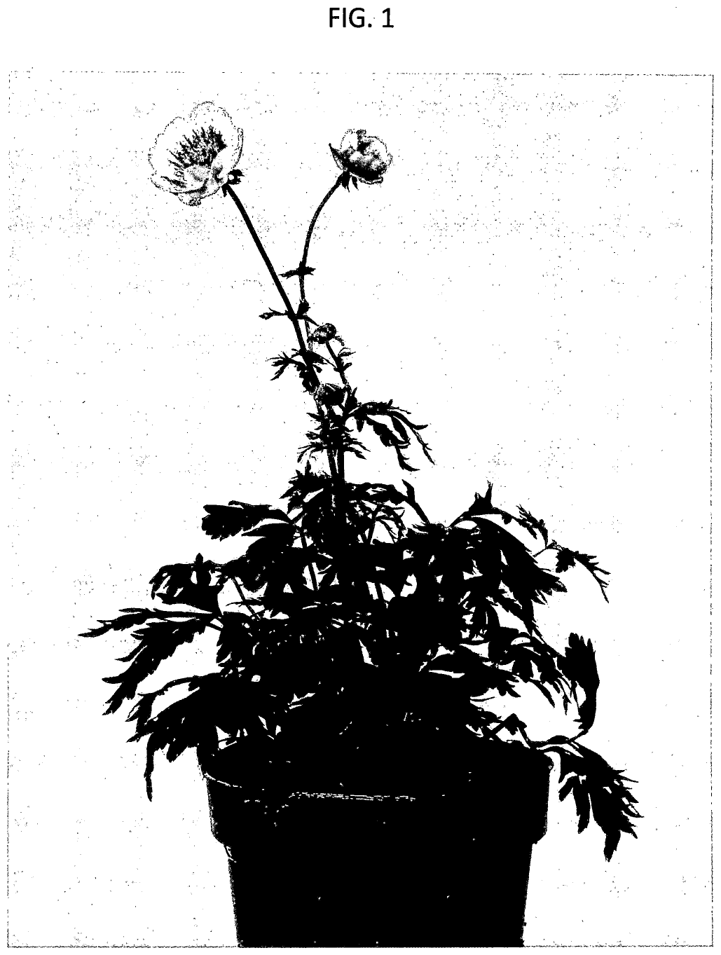

FIG. 2 shows, as nearly true as it is reasonably possible to make the same in color illustrations of this type, the typical mature foliage of ‘MACTRO010’.

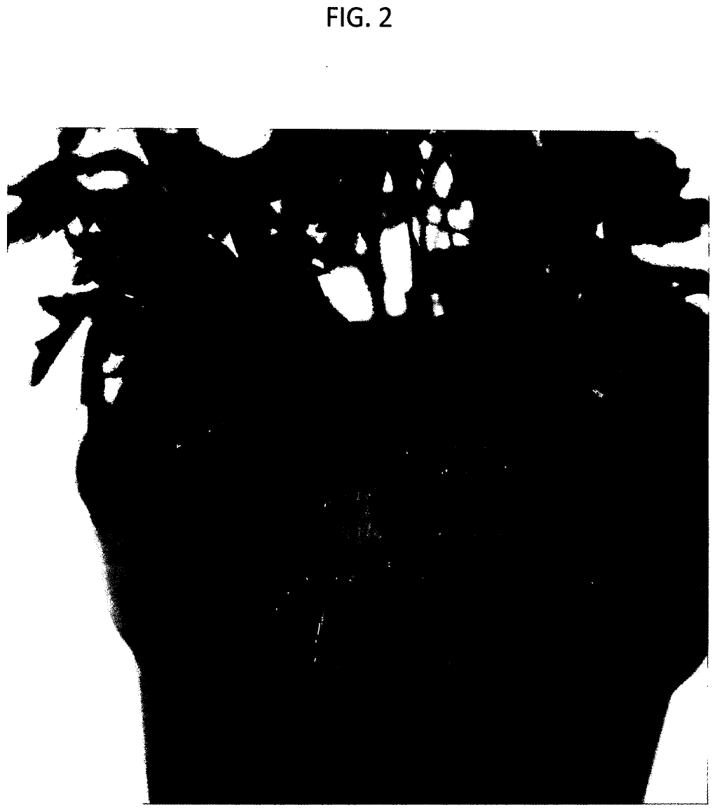

FIG. 3 shows, as nearly true as it is reasonably possible to make the same in color illustrations of this type, the typical flower of ‘MACTRO010’.

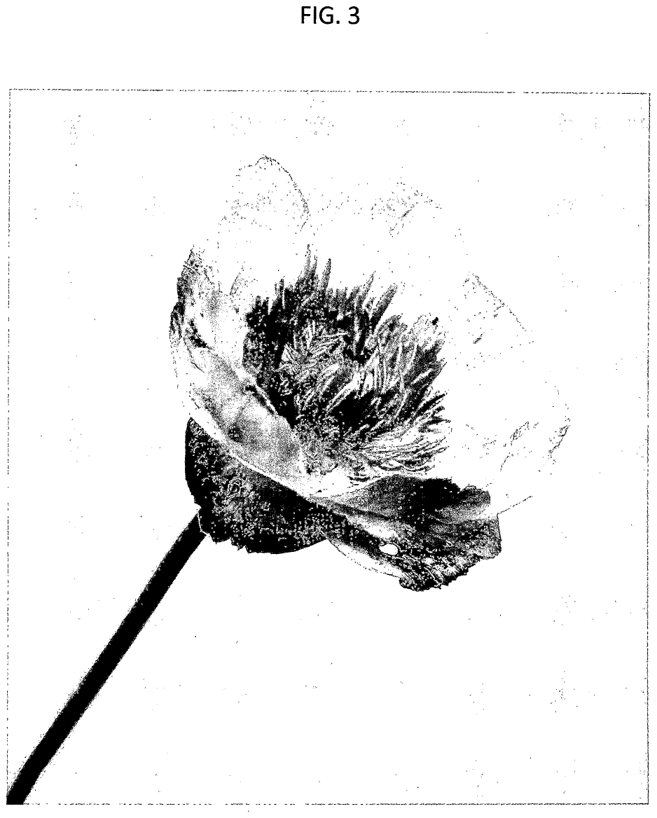

### BOTANICAL DESCRIPTION OF THE PLANT

The following is a detailed botanical description of a new and distinct variety of Trollius plant known as ‘MACTRO010’, based upon observations of 15-month-old plants grown in 19 cm nursery containers in the Netherlands. Plants were grown outdoors, in full sun, from spring until early fall and then moved into a heated greenhouse in October of 2023 to promote flowering. From spring until the plants were moved into a greenhouse, daytime high temperatures ranged from 10 to 20 degrees Celsius and nighttime temperatures ranged from 4 to 15 degrees Celsius. Plants were watered as needed and fertilized with liquid fertilizer at regular intervals. No chemical pest treatments were given to the plants. Observation data was recorded in November of 2023.

A botanical description of ‘MACTRO010’ and comparisons with the parent and most similar variety of common knowledge are provided below.
General plant description:
   Growth habit.—Herbaceous perennial; globular plant habit with leaves developing from basal rosettes; flowering stems arising from the rosette also bear foliage with flowers borne above the foliar plane; no lateral branching is present.
   Plant profile.—Flattened globular to globular.
   Height.—27.3 cm to the top of the foliar plane; 37.0 cm to the top of the floral plane.
   Spread.—30.5 cm.
   Plant vigor.—Moderately vigorous.
   Growth rate.—Moderately growing.
   Propagation.—Method — Meristematic tissue culture. Time to initiate rooting — Approximately 8 to 10 weeks at an average ambient temperature of 20 degrees Celsius. Time to produce flowering plant from rooted cutting — Approximately 36 to 40 weeks in a 15 cm container.
   Pest resistance and susceptibility.—Plants have not been observed to be any more or less susceptible or resistant to pathogens and pests known to affect Trollius sp.
   Environmental tolerances.—Adapt to, at least, USDA Zones 4 to 9 and temperatures ranging from minus 30 degrees Celsius to 35 degrees Celsius; moderate to high tolerance to rain; moderate tolerance to wind.
Stem:
   Branching habit.—Unbranched flowering stems arise from the basal rosette in spring.
   Number of primary (main) stems per plant.—4.
   Number of secondary (lateral) branches per plant.—4.
   Attitude.—Near vertical; at an average angle of 15 degrees from vertical.
   Aspect.—Elliptic.
   Length.—24.1 cm, from the base to the distalmost leaf.
   Diameter.—0.4 cm on average.
   Internode length.—6.8 cm.
   Texture.—Smooth and glabrous.
   Luster.—Moderately glossy.
   Strength.—Strong.
   Color, juvenile.—Yellow-green, nearest to RHS 144A.
   Color, mature.—Green, nearest to RHS 143B.
   Color at internodes.—Green, nearest to RHS 143B.
Foliage:
   Arrangement.—Leaves are borne both basally and on the flowering stems.
   Basal leaves.—Attachment — Petiolate; sheathed. Sheaths — Length — 2.5 cm. Width — 1.7 cm. Color — White, nearest to RHS 155C. Phyllotaxy — Alternate. Division — Trifoliate. Compound leaf outline — Ovate to near orbicular. Length — 11.2 cm, excluding the petiole. Width — 12.5 cm. Attitude — Outward to pendulous. Leaflets — Shape — The terminal leaflets are elliptic and lobed, while the lateral leaflets are broad ovate and deeply lobed to near-cleft. Lobes — Depth of sinuses — Deep to very deep. Sinus orientation — Divergent. Length, terminal leaflet — 8.2 cm. Width, terminal leaflet — 7.3 cm. Length, lateral leaflets — 4.2 cm. Width, lateral leaflets — 6.8 cm. Attitude — Outward, relative to the petiole. Aspect — Relatively flat; occasional concave or convex. Apex — Acute to narrowly acute. Base — Attenuate. Margin — Coarsely serrated. Texture, adaxial surface — Smooth and glabrous. Texture, abaxial surface — Smooth and slightly coriaceous. Luster, adaxial surface — Slightly glossy. Luster, abaxial surface — Very slightly glossy. Color — Juvenile foliage color, adaxial surface — Yellow-green, nearest to a mixture of RHS 144A and 144B. Juvenile leaf color, abaxial surface — Yellow-green, nearest to a mixture of RHS 145A and 146D. Mature leaf color, adaxial surface — Green, nearest to RHS NN137B. Mature leaf color, abaxial surface — Yellow-green, nearest to a mixture of RHS 147B and 148B. Venation — Type — Laciniate. Vein color, adaxial surface — Green, nearest to in between RHS 143A and 143B. Vein color, abaxial surface — Yellow-green, nearest to RHS 144A. Petiole — Length — 12.5 cm. Diameter — 0.25 cm. Strength — Strong. Texture, adaxial and abaxial surfaces — Smooth and glabrous. Luster, adaxial and abaxial surfaces — Moderately glossy to glossy Color, adaxial surface — Nearest to a mixture of green and yellow-green, RHS 143B and 144A. Color, abaxial surface — Yellow-green, RHS 144A.
   Leaves bome on the stems.—Attachment — Petiolate. Phyllotaxy — Alternate. Division — Trifoliate. Compound leaf outline — Ovate to near orbicular. Length — 6.9 cm, excluding the petiole. Width — 8.1 cm. Attitude — Outward to pendulous. Leaflets — Shape — The terminal leaflets are elliptic and lobed, while the lateral leaflets are broad ovate and deeply lobed to near-cleft. Lobes — Depth of sinuses — Deep to very deep. Sinus orientation — Divergent. Length, terminal leaflet — 5.2 cm. Width, terminal leaflet — 4.5 cm. Length, lateral leaflets — 2.6 cm. Width, lateral leaflets — 3.3 cm. Attitude — Outward, relative to the petiole. Aspect — Relatively flat; occasional concave or convex. Apex — Acute to narrowly acute. Base — Attenuate. Margin — Coarsely serrated. Texture, adaxial surface — Smooth and glabrous. Texture, abaxial surface — Smooth and slightly coriaceous. Luster, adaxial surface — Slightly glossy. Luster, abaxial surface — Very slightly glossy. Color — Juvenile foliage color, adaxial surface — Yellow-green, nearest to a mixture of RHS 144A and 144B. Juvenile leaf color, abaxial surface — Yellow-green, nearest to a mixture of RHS 145A and 146D. Mature leaf color, adaxial surface — Green, nearest to RHS NN137B. Mature leaf color, abaxial surface — Yellow-green, nearest to a mixture of RHS 147B and 148B. Venation — Type — Laciniate. Vein color, adaxial surface — Green, nearest to in between RHS 143A and 143B. Vein color, abaxial surface — Yellow-green, nearest to RHS 144A. Petiole — Length — 12.5 cm. Diameter — 0.25 cm. Strength — Strong. Texture, adaxial and abaxial surfaces — Smooth and glabrous. Luster, adaxial and abaxial surfaces — Moderately glossy to glossy. Color, adaxial surface — Nearest to a mixture of green and yellow-green, RHS 143B and 144A. Color, abaxial surface — Yellow-green, RHS 144A.
Inflorescence: No inflorescences; flowers are solitary.
Flower buds:
   Shape.—Flattened globular.
   Length.—1.0 cm.
   Diameter.—1.7 cm.
   Texture.—Smooth and glabrous.
   Luster.—Slightly glossy.
   Color.—Yellow-green, nearest to RHS 144C.
Flower:
   General description.—Perianths do not exhibit separate sepals and petals but rather several outer whorls of large, inwardly-cupped tepals which envelope the tepaloids which themselves ring the androecium and gynoecium.
   Natural flowering season.—Late spring through late summer.
   Quantity of flowers and flower buds per plant.—5, as observed.
   Shape, type.—Cyanthiform; single.
   Floriferousness.—Low floriferousness to moderately floriferous.
   Attitude.—Upright to slightly outward.
   Flower longevity on plant.—Approximately 10 days.
   Persistent or self-cleaning.—Self-cleaning.
   Fragrance.—Non-fragrant.
   Time to flowering response.—Approximately 10 months from potting.
   Diameter.—5.5 cm.
   Depth.—2.9 cm.
   Pedicels.—Length — 10.1 cm. Diameter — 0.25 cm. Angle — Average angle to stem axis is 17.5 degrees on average. Strength — Moderately strong. Texture — Smooth, striated, and glabrous. Luster — Moderately glossy. Color — Green, nearest to in between RHS 143A and 143B.
   Tepals.—Quantity — Ranging from 10 to 13; 12 on average. Arrangement — Rotate; arranged into four whorls of unfused tepals. Attitude — Upright. Aspect — Strongly concaved. Length — 3.1 cm. Width — 2.2 cm. Shape — Obovate to broad obovate. Apex — Obtuse to cuneate. Base — Cuneate. Margin — Entire; lightly undulated to no undulation. Texture, upper surface — Glabrous, slightly velvety. Texture, lower surface — Glabrous and slightly velvety. Luster, upper surface — Matte. Luster, lower surface — Very slightly velvety. Tepal color — When opening, upper surface — Green-white, nearest to RHS 157B, and fading to yellow-green distally, nearest to RHS 150D. When opening, lower surface — Green-white, nearest to RHS 157B, and fading to yellow-green distally, nearest to RHS 150D. Outermost tepals are yellow-green, nearest to RHS 150D, and slightly darker distally, nearest to RHS 154C. Fully opened, upper surface — Green-white, nearest to RHS 157D, and becoming slightly darker distally, nearest to in between RHS 157B and 157C. Tepal color does not fade with age. Fully opened, lower surface — Green-white, nearest to RHS 157B, and fading to yellow-green distally, nearest to RHS 150D. Outermost tepals are yellow-green, nearest to RHS 150D, and slightly darker distally, nearest to RHS 154C. Tepal color does not fade with age. Venation color, fully opened, upper surface — No veins are visible. Venation color, fully opened, lower surface — Main veins are yellow-green, nearest to in between RHS 145C and 145D; no other veins are visible, with the exception of the outermost tepal whorl. The outermost tepals are veined yellow-green, nearest to RHS 145B.
   Tepaloids.—Quantity — 16. Arrangement — Rotate; arranged in a single whorl of unfused tepaloids. Attitude — Upright. Aspect — Flat. Length — 1.8 cm. Width — 0.2 cm. Shape — Linear. Apex — Bluntly acute. Base — Narrowly cuneate. Margin — Entire; no undulation. Texture, upper surface — Smooth and glabrous. Texture, lower surface — Smooth and glabrous. Luster, upper surface — Very slightly velvety. Luster, lower surface — Matte. Tepaloid color — When opening, upper surface — Yellow-green, nearest to RHS 150B, and fading to a slightly lighter shade of yellow-green distally, nearest to RHS 150C; base is green-white, nearest to in between RHS 157C and 157D. A very small, brown nectary is present near the base of the tepaloids, nearest to RHS N200A. When opening, lower surface — Yellow-green, nearest to RHS 150B, and fading to a slightly lighter shade of yellow-green distally, nearest to RHS 150C; base is green-white, nearest to in between RHS 157C and 157D. A very small, brown nectary is present near the base of the tepaloids, nearest to RHS N200C. Fully opened, upper surface — Yellow-green, nearest to RHS 151C, and fading to a slightly lighter shade of yellow-green distally, nearest to RHS 153D; base is green-white, nearest to in between RHS 157C and 157D. A very small, brown nectary is present near the base of the tepaloids, nearest to RHS 200A. Tepaloid color does not fade with age. Fully opened, lower surface — Yellow-green, nearest to RHS 150A, and fading to a slightly lighter shade of yellow-green distally, nearest to RHS 151C; base is green-white, nearest to in between RHS 157C and 157D. A very small, brown nectary is present near the base of the tepaloids, nearest to RHS N200C. Tepaloid color does not fade with age. Venation color, fully opened, upper surface — No veins are visible. Venation color, fully opened, lower surface — No veins are visible.
Reproductive organs:
   Androecium.—Stamens — Quantity — Approximately 180. Anthers — Attachment — Basifixed. Shape — Narrowly oblong. Dimensions — 0.4 cm long and 0.1 cm wide. Color — Yellow-green, nearest to RHS 154B. Filaments — Length — 0.9 cm. Color — White, nearest to NN155B. Amount of Pollen — Moderately abundant. Pollen color — White, RHS 155B.
   Gynoecium.—Pistil — Quantity — 55. Length — 0.15 cm. Style — Length — 0.1 cm. Color — Yellow-green, nearest to RHS 154D. Stigma — Shape — Club-shaped; curled downward. Length — 0.1 cm. Diameter — 0.3 cm. Color — Yellow-green, nearest to RHS 154D. Ovary Color — Yellow-green, nearest to RHS 146D.
Seed and fruit: No seeds or fruit have been observed.
Comparisons with the parent plants: Plants of the new cultivar ‘MACTRO010’ differ from its seed parent, Trollius ‘Cheddar’ (not patented), by the characteristics described in Table 1.

TABLE 1

| Characteristic | ‘MACTRO010’ | ‘Cheddar’ |
|---|---|---|
| Flower size. | Larger than ‘Cheddar’. | Smaller than |
| | | ‘MACTRO010’. |
| Quantity | More than ‘Cheddar’. | Fewer than |
| of tepals. | | ‘MACTRO010’. |
| General | Yellow-green, | Light yellow; darker |
| coloration | yet generally | than ‘MACTRO010’. |
| of the | appearing as | |
| tepaloids. | a lighter shade | |
| | of yellow relative | |
| | to ‘Cheddar’. | |
| General | Yellow-green, | Light yellow; darker |
| coloration | yet generally | than ‘MACTRO010’. |
| of the | appearing as | |
| androecium. | a lighter shade | |
| | of yellow relative | |
| | to ‘Cheddar’. | |

Comparison with the most similar variety of common knowledge: Plants of the new cultivar ‘MACTRO010’ may be distinguished from the most similar known commercial comparator, Trollius ‘Alabaster’ (not patented), by the characteristics described in Table 2.

TABLE 2

| Characteristic | ‘MACTRO010’ | ‘Alabaster’ |
|---|---|---|
| Plant height. | Taller than ‘Alabaster’. | Shorter than |
| | | ‘MACTRO010’. |
| Flower size. | Larger than ‘Cheddar’. | Smaller than |
| | | ‘MACTRO010’. |
| Quantity | More than ‘Cheddar’. | Fewer than |
| of tepals. | | ‘MACTRO010’. |
| General | Yellow-green, yet | Light primrose yellow. |
| coloration | generally appearing as | |
| of the flower. | a pale cream yellow | |
| | coloration. | |

That which is claimed is:

1. A new and distinct variety of Trollius plant named ‘MACTRO010’, substantially as described and illustrated herein.
